(19) United States
(12) Patent Application Publication
(10) Pub. No.: US 2015/0290038 A1
(43) Pub. Date: Oct. 15, 2015

(54) Goggle Lens Positioning Structure

(71) Applicant: Jiann Lih Optical Co., Ltd., Tainan City 717 (TW)

(72) Inventor: Chen-Chang Wang-Lee, Tainan City (TW)

(21) Appl. No.: 14/336,021

(22) Filed: Jul. 21, 2014

(30) Foreign Application Priority Data
Apr. 14, 2014 (TW) 103113570

(51) Int. Cl. A61F 9/02

(52) U.S. Cl. CPC A61F 9/029; A61F 9/026

(57) ABSTRACT

A goggle lens positioning structure includes a protective soft pad, a frame, and a lens. The lens and the frame are coupled together by following the steps below. Pressing portions and carrying portions disposed at upper and lower sides of the frame and extending forward are inserted into concave portions concavely disposed at corresponding portions of the lens. After penetrating holes disposed at corresponding portions of the lens, branch portions extending from two outer sides of the frame leave a hole at one end of a fastening unit to restrict movement. The frame is received in the receiving space of the protective soft pad to effectuate engagement therebetween. Hence, the lens and the frame can be quickly put together and easily separated.

### BACKGROUND OF THE INVENTION

1. Technical Field

The present invention relates to goggle lens positioning structures, and more particularly, to a goggle lens positioning structure whose related components can be inserted into each other by vertical engagement or by a predetermined segment thereof, wherein not only are the related components of the goggle lens positioning structure engaged with each other precisely when manipulated quickly, but the coupling portions of the related components also remain undamaged when dismounted.

2. Description of Related Art

Conventional eyeglasses have wide applications, including eyesight correction, sports, and preventing intense light from injuring the eye. The conventional eyeglasses are available in different categories for selection by users. The present invention is intended to improve the conventional sports eyeglasses. The conventional sports eyeglasses essentially comprise two integrally-formed lenses. The junction of the two integrally-formed lenses is coupled to a protective frame. The lateral sides of the two integrally-formed lenses are coupled to a cord disposed around the user's head comfortably. Hence, the conventional sports eyeglasses are safe and comfortable to wear.

U.S. Pat. No. 7,725,959 B1, entitled Protective Goggles and issued to the inventor of the present invention on Jun. 1, 2010, discloses a pair of protective goggles comprising: an integrally-formed lens, wherein an opening is formed at a middle segment of an upper side of the lens for correspondingly engaging with an engaging portion on an upper side of the frame having a protective pad made of soft materials; and a protrusion extending upwardly from an inner surface of a nose supporting portion at a lower side of the lens to be correspondingly inserted into a hole adjacent to a concave segment formed on a middle segment of a lower side of the frame for positional restriction; wherein the frame further has more than one rib extending on an upper side of the frame for correspondingly fitting with the lens and for stably propping the lens, wherein a through hole is provided on each end of the frame and has a raised portion projected around the through hole, whereas a tenon is formed and is adjacent to the raised portion and passing through an aperture of the lens, with the raised portion projecting through the corresponding through hole of the lens; and a cord having two ends respectively combined with fastening units for penetration and change in direction such that the cord is fastened integrally to the fastening units.

The present invention aims to provide a novel design based on the configuration of related components of a goggle lens positioning structure which falls within the same category as the aforesaid protective goggles. The goggle lens positioning structure comprises related components, such as a protective soft pad, a frame, and a lens, which can be inserted into each other by vertical engagement or by a predetermined segment thereof, such that not only are the related components of the goggle lens positioning structure engaged with each other precisely when manipulated quickly, but the coupling portions of the related components also remain intact and undamaged when dismounted.

### BRIEF SUMMARY OF THE INVENTION

The present invention provides a novel goggle lens positioning structure based on the configuration of components of conventional protective goggles. The goggle lens positioning structure comprises a protective soft pad, a frame, and a lens and is characterized in that: pressing portions and carrying portions formed at upper and lower portions of the frame and extending forward are inserted into a concave portion concavely disposed at the lens; branch portions extend from two outer sides of the frame penetrate holes at corresponding portions of the lens to leave holes at the ends of fastening units so as to effectuate positional restriction; the frame is received in a receiving space disposed at the protective soft pad, such that two components effectuate engagement therebetween, enabling the quick positioning of the lens and the frame relative to each other, and enabling easy disassembly.

It is an objective of the present invention to provide a goggle lens positioning structure, comprising a protective soft pad, a frame, a lens, and two fastening units for use with a cord, characterized in that: pressing portions and carrying portions are disposed on upper and lower sides of a middle segment of the frame, respectively, such that a concave portion concavely disposed at an upper end of the lens is pushed to hold the pressing portions, whereas a concave portion concavely disposed at a lower end of the lens straddles the carrying portions, wherein branch portions extending from two outer sides of the frame leave holes on two sides of the lens to leave a hole at an end of the fastening units, thereby effectuating a positional restriction of the lens and the frame, wherein the frame coupled to the lens is received in a receiving space disposed at the protective soft pad to allow the branch portions on the two sides of the frame to press against the concave portions at the protective soft pad, enable engagement between the frame and related components of the goggle lens positioning structure, enable the quick positioning of the lens and the frame relative to each other, and enable easy disassembly.

Another objective of the present invention is to provide the goggle lens positioning structure, wherein a lower portion of the frame received in the receiving space disposed at the protective soft pad extends forward to form a rod for replacing the carrying portions and thereby penetrating the hole disposed at the lens, so as to replace the straddling of the concave portions, thereby effectuating quick positioning of the lens and the frame.

Yet another objective of the present invention is to provide the goggle lens positioning structure, wherein a protrusion slightly larger than the periphery thereof is disposed at an outer portion of a rod formed by extending forward a lower portion of the frame received in the receiving space disposed at the protective soft pad, thereby effectuating a positional restriction when penetrating the holes of the lens.

### BRIEF DESCRIPTION OF THE SEVERAL VIEWS OF THE DRAWINGS

FIG. 1 is an exploded view of a goggle lens positioning structure according to the present invention;

FIG. 2 is a perspective view of the goggle lens positioning structure according to the present invention;

FIG. 2A is a transverse cross-sectional view of portion A shown in FIG. 2;

FIG. 3 is a longitudinal cross-sectional view of a middle segment of the goggle lens positioning structure according to the present invention;

FIG. 4 is an exploded view of the goggle lens positioning structure according to another embodiment of the present invention; and

FIG. 5 is a longitudinal cross-sectional view of the middle segment of the goggle lens positioning structure according to another embodiment of the present invention.

### DETAILED DESCRIPTION OF THE INVENTION

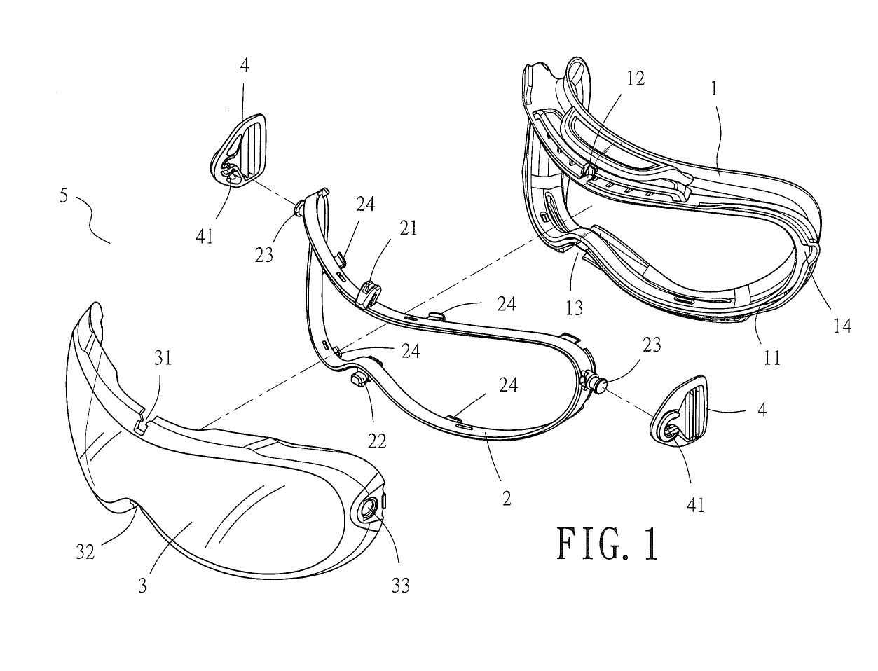

The present invention provides a goggle lens positioning structure. Referring to FIG. 1, a goggle 5 comprises a protective soft pad 1, a frame 2, a lens 3, and two fastening units 4 for use with a cord.

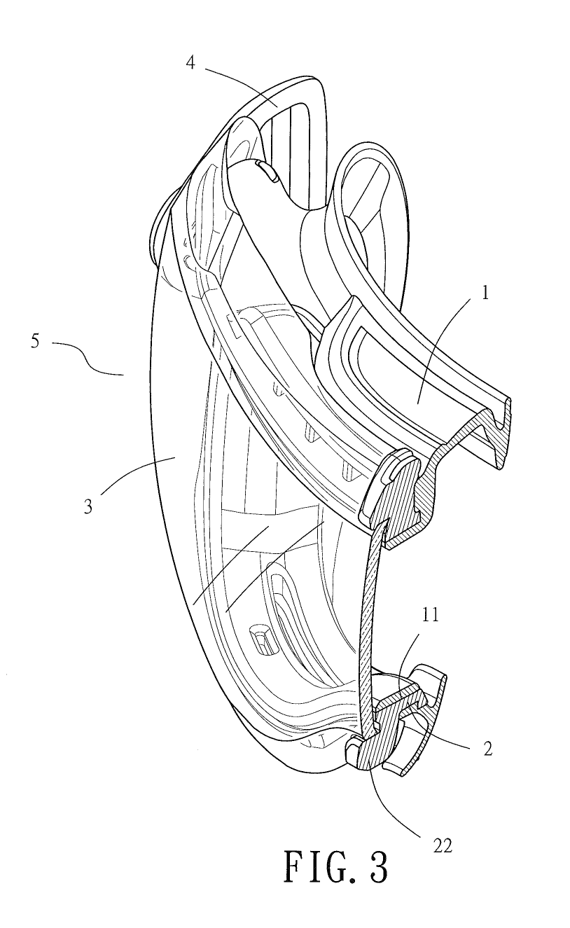

A receiving space 11 is circumferentially disposed at the periphery of the protective soft pad 1 as shown in FIG. 3. Openings 12, 13 opening forward are disposed concavely at the protective soft pad 1 and positioned proximate to the outer periphery of the receiving space 11, and correspond in position to the upper and lower portions thereof. A concave portion 14 opening outward is disposed concavely at the protective soft pad 1 and corresponds in position to the two sides of the receiving space 11.

Components appropriately connectable to the frame 2 are selected according to the receiving space 11 circumferentially disposed at the periphery of the protective soft pad 1. Pressing portions 21 and carrying portions 22 are formed on upper and lower sides of a middle segment of the frame 2, respectively. Branch portions 23 extend from two outer sides of the frame 2, respectively. At least a hook portion 24 extends backward from the upper and lower portions of the frame 2. The at least a hook portion 24 bends to form an L-shaped pressing edge facing sideward, such that the at least a hook portion 24 of the frame 2 presses against the periphery of the receiving space 11 steadily (not shown).

The lens 3 is selected according to the configuration of the peripheries of the frame 2 and the protective soft pad 1. Concave portions 31, 32 are concavely disposed on the upper and lower sides of a middle segment of the lens 3, respectively. Holes 33 of appropriate shape and size are disposed on two sides of the lens 3, respectively.

The two fastening units 4 each have an end with a hole 41 and another end with a groove which a cord winds around.

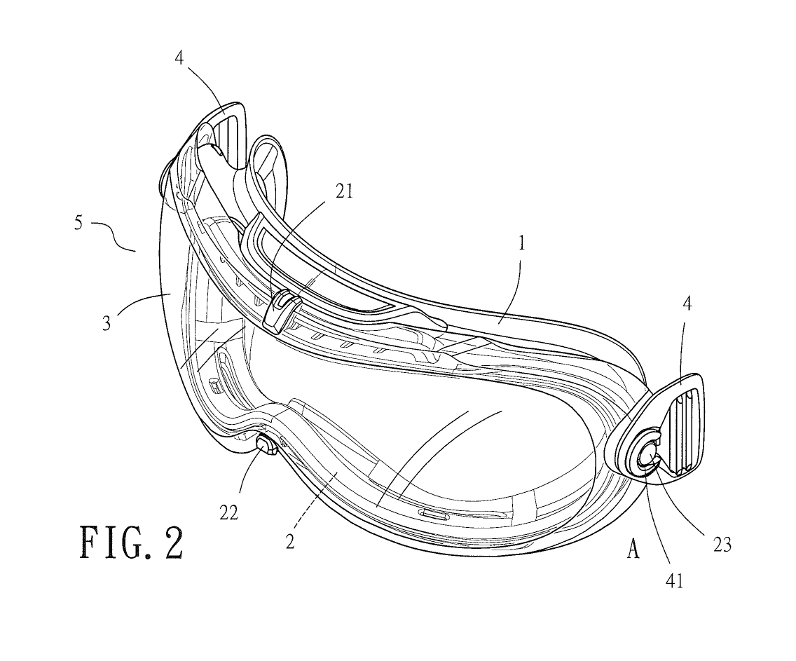

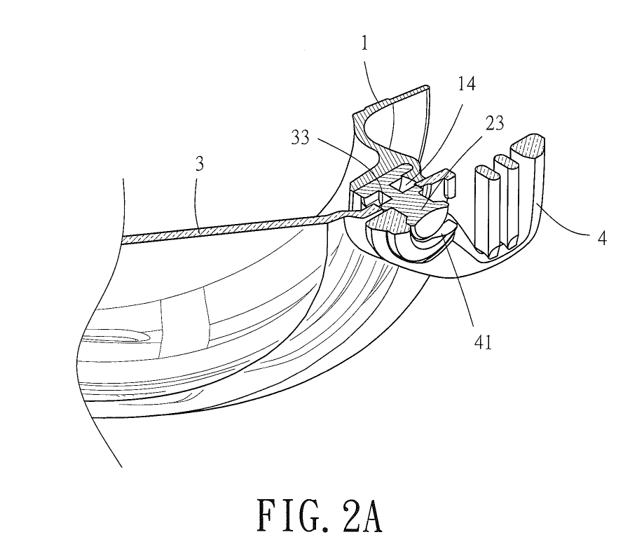

Three steps of the process flow of effectuating engagement between the protective soft pad 1, the frame 2, the lens 3, and the fastening units 4 are illustrated with FIG. 1 and FIG. 2. The first step involves pushing the concave portion 31 at an upper end of the lens 3 to hold the pressing portions 21 of the frame 2 and positioning the concave portion 32 at a lower end of the lens 3 in a manner that the concave portion 32 straddles the carrying portion 22 at the lower end of the frame 2 as shown in FIG. 3. The second step involves passing the branch portions 23 extending from two outer sides of the frame 2 through the holes 33 on two sides of the lens 3 and then allowing the branch portions 23 to leave the holes 41 at respective ends of the fastening units 4 as shown in FIG. 2A, thereby effectuating a positional restriction of the lens 3 and the frame 2. The third step involves fitting the frame 2 coupled to the lens 3 into the receiving space 11 disposed at the protective soft pad 1 to allow the branch portions 23 on two sides of the frame 2 to press against the concave portions 14 disposed at the protective soft pad 1 as shown in FIG. 2 and enable engagement between the frame 2 and related components of the goggle 5, such that not only are the lens 3 and the frame 2 quickly positioned relative to each other when operated in a simple manner, but the coupling portions of the related components also remain intact and undamaged when dismounted, not to mention that the related components can be easily dismounted.

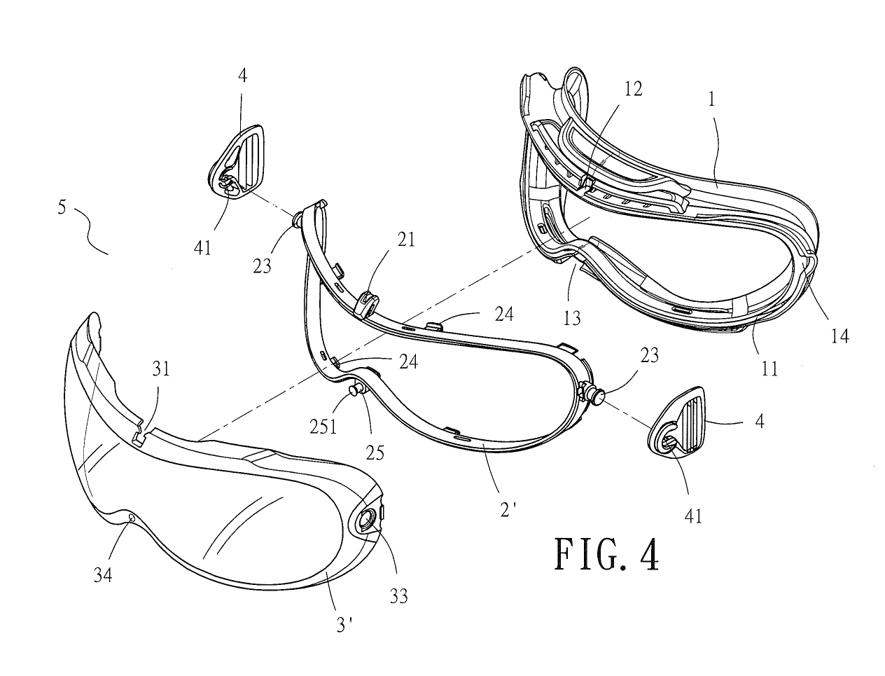

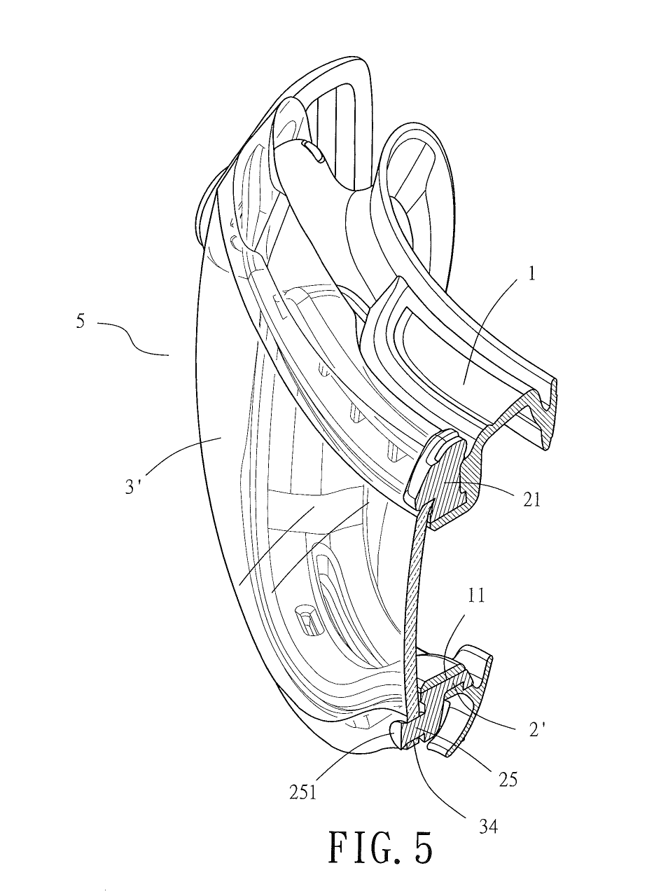

Referring to FIG. 4, the second embodiment of a lens 3′ and a frame 2′ of the goggle 5 is identical to the first embodiment in terms of components, which include a protective soft pad 1, a frame 2′, a lens 3′, and the two fastening units 4 for use with a cord, except that, in the second embodiment, a lower portion of the frame 2′ received in the receiving space 11 disposed at the protective soft pad 1 extends forward to form a rod 25 for replacing the carrying portions; and a protrusion 251 slightly larger than the periphery thereof is disposed at an outer portion of the rod 25, so as to penetrate a hole 34 disposed at the lens 3′, as shown in FIG. 5, and thus replace the straddling of the concave portions, thereby effectuating the quick positioning of the lens 3′ and the frame 2′.

What is claimed is:

1. A goggle lens positioning structure, comprising a protective soft pad, a frame, a lens, and two fastening units for use with a cord, characterized in that: pressing portions and carrying portions are disposed on upper and lower sides of a middle segment of the frame, respectively, such that a concave portion concavely disposed at an upper end of the lens is pushed to hold the pressing portions, and a concave portion concavely disposed at a lower end of the lens straddles the carrying portions, wherein branch portions extending from two outer sides of the frame leave holes on two sides of the lens to leave a hole at an end of the fastening units, thereby effectuating a positional restriction of the lens and the frame,
wherein the frame coupled to the lens is received in a receiving space disposed at the protective soft pad to allow the branch portions on the two sides of the frame to press against the concave portions at the protective soft pad, enable engagement between the frame and related components of the goggle lens positioning structure, enable quick positioning of the lens and the frame relative to each other, and enable easy disassembly.

2. The goggle lens positioning structure of claim 1, wherein a lower portion of the frame received in the receiving space disposed at the protective soft pad extends forward to form a rod for replacing the carrying portions and thereby penetrating the hole disposed at the lens, so as to replace the straddling of the concave portions, thereby effectuating quick positioning of the lens and the frame.

3. The goggle lens positioning structure of claim 2, wherein a protrusion slightly larger than a periphery thereof is disposed at an outer portion of a rod formed by extending forward a lower portion of the frame received in the receiving space disposed at the protective soft pad, thereby effectuating a positional restriction when penetrating the holes of the lens.
